(19) United States
(12) Patent Application Publication
(10) Pub. No.: US 2005/0164199 A1
(43) Pub. Date: Jul. 28, 2005

(54) Method for biochemical analysis of dna and arrangement associated therewith

(76) Inventors: Manfred Stanzel, Erlangen (DE); Mathias Sprinzl, Bayreuth (DE)

Correspondence Address: HARNESS, DICKEY & PIERCE, P.L.C., P.O.BOX 8910, RESTON, VA, 20195, US

(21) Appl. No.: 10/514,019

(22) PCT Filed: May 18, 2003
(86) PCT No.: PCT/DE03/01479

(30) Foreign Application Priority Data
May 20, 2002 (DE) 10220935.9

(51) Int. Cl. C12Q 1/68

(52) U.S. Cl. 435/6

(57) ABSTRACT

A system with immobilized DNA is used in the fields of medicine, environmentology or criminology as an analytical tool in the analysis of nucleic acid. The immobilized DNA is provided with a biocatalytically active marker, such as an enzyme, an with an inhibitor substance which reversibly inhibits catalytic activity, or in addition to the immobilized biocatalytic marker, the immobilized DNA is provided with a substance which can reversibly inhibit the catalytic activity of the marker. Alternatively, an immobilized biocatalytically active marker can be provided with DNA as a scavenger which includes a substance as an inhibitor which can reversibly inhibit the activity of the marker. In another alternative, it is possible to use a complex including a molecule binding double-stranded DNA and a substance as an inhibitor which can reversibly inhibit the activity of the marker by interacting with the immobilized biocatalytically active marker. In all cases, the inhibitor or compound including an inhibitor and a molecule which can bind double-stranded DNA interacts with the biocatalytically active marker and defines the inactive state of the system. When the DNA, which is to be analyzed, is bonded, especially hybridized, to the DNA scavengers, the interaction between the biocatalytically active marker and the inhibitor is cancelled as a result of the formation of the double strand. The system is thus shifted from a first state into a second state defining the active state. A carrier with integrated microelectrodes is provided in the associated device, whereby the enzyme is either immobilized therein or is contained in a polymer network in the vicinity of the microelectrodes.

This application is the national phase under 35 U.S.C. § 371 of PCT International Application No. PCT/DE03/01479 which has an International filing date of May 8, 2003, which designated the United States of America and which claims priority on German Patent Application number DE 102 20 935.9 filed May 10, 2002, the entire contents of which are hereby incorporated herein by reference.

### FIELD OF THE INVENTION

The invention generally relates to a method for biochemically analyzing DNA. In addition, the invention generally relates to an associated arrangement for implementing this method.

The fields of application of the invention are, in particular, medicine, environmental analysis and forensics. In this connection, an enzyme, as an immobilized biocatalytically active label, an immobilized DNA and an immobilized substance (inhibitor), which is able to inhibit the activity of the enzyme reversibly, are used as tools for analyzing nucleic acids.

In the present context, DNA (deoxyribonucleic acid) is understood as meaning a deoxyribonucleic acid and its structural analogs. These are, in particular, PNA (peptide nucleic acid), “caged” DNA, RNA (ribonucleic acid) and all 2′-substituted DNA derivatives.

The invention is not applicable to ribozymes. In this connection, the reader is referred to the publication “Catalytic Molecular Beacons” in CHEMBIOCHEM 2001, 2, 411-415.

The aim of the present developments is that of performing a molecular analysis at the level of DNA and/or gene expression, in the latter case by way of analyzing cDNA (complementary DNA) in particular. This makes it possible to identify and type hereditary material-containing pathogens, such as bacteria and viruses or the like, and to clarify any resistances which may be present. Furthermore, it makes it possible to detect organisms in environmental analysis, foodstuffs technology and agriculture. In addition to this, the use of this type of DNA analysis in medicine offers the possibilities of rapidly performing hereditary disease, predisposition and/or tumor diagnosis as well as monitoring therapy.

### BACKGROUND OF THE INVENTION

In accordance with the prior art, immobilized DNA is used as an analytical tool in analyzing the sequences of nucleic acids. For this, synthetic DNA having a length of up to 100 nucleotide building blocks (DNA oligonucleotides) is covalently linked to a suitable surface by way of an active group. The surfaces which are used can be silicates, metal layers, for example gold or the like, or else a variety of polymer layers.

The latter technology is highly developed and makes it possible to specifically immobilize DNA oligo-nucleotides of defined sequence on areas having a diameter of up to a few μm or in volumes having a content of up to a few nl. Complementary DNA molecules from the sample can then be bound to these areas or volumes which are occupied by what are termed the DNA catchers. The specificity of this binding is defined by the rule of DNA complementary base pairing. When a range of DNA molecules having different sequences is present in the analyte solution, those DNA molecules which conform best with the base pairing rules, and which release the greatest quantity of energy in connection with the complex formation, will bind to the catcher.

Specific selection, what is termed stringency, of the external conditions, such as temperature, ionic strength, etc., during the binding by means of hybridization results in only the most stable pairings of catcher and analyte DNA, that is those pairings which conform completely to the base pairing rules, being selectively retained.

The latter is the basis of DNA analysis on what are termed DNA chips. The general advantages of such a DNA analysis on chips are to be seen in the high degree of miniaturization, the synchronization and the high speed of the overall process as compared with conventional methods. Because of the lower requirement for reagents and sample material, this is accompanied by a reduction in costs. In addition to this, the use of DNA chips leads to an increase in the efficiency and precision of the DNA analysis process.

The various types of DNA chip differ, in particular, in the choice of the substrate, such as plastic, glass, silicon, etc., in the method of immobilization, e.g. gold-thiol coupling, immobilization in gel or the like, in the technology of the application to the solid surface, such as on-line synthesis, dispensing or the like, and in the nature of the detection, in particular optical and/or electrochemical, of the DNA interactions.

The spectroscopic systems in which the DNA to be analyzed is provided, by means of PCR (polymerase chain reaction) or SDA (strand displacement amplification), with a fluorescent reporter group, as explained diagrammatically by means of FIG. 1, are the most widespread. In detail, the circles in the figure represent the spectroscopic reporter groups which are coupled to the analyte DNA either directly, by way of PCR/SDA, or indirectly, by way of what are termed fluorescent signal oligonucleotides which have been introduced in what is termed a sandwich hybridization assay. After the analyte DNA which does not bind, or only binds weakly, has been removed by applying what are termed stringent conditions, e.g. high temperature, low ionic strength, organic solvent, the sites at which the interaction has taken place can be visualized by means of fluorescence microscopy, junction-type detectors or a CCD camera. The regions on the surface at which the interaction between catcher and analyte DNA has taken place appear as spots which possess altered optical properties. Since the positions of the different catcher DNAs on the chip are known, the corresponding complementary DNA which is present in the analyte samples can be identified unambiguously.

DNA chips containing some thousand different oligonucleotides/cm² are commercially available, as are systems for optical analysis. In particular, EP 0 745 690 A2 describes optical systems containing probes in which what are termed stem loop structures are refolded by hybridization, with this being detected optically.

In the case of optical detection systems, comparatively complicated reading and analytical instruments are required, with these instruments immediately primarily restricting the use of the DNA chip technology to specialized laboratories. It is doubtful whether the DNA analysis of this type can be applied broadly in field analysis, e.g. in agricultural businesses, in the foodstuffs industry, in environmental analysis or in production-accompanying analysis, or in the case of doctors having their own independent practices. Simply preparing the samples using PCR or SDA and/or introducing the spectroscopic reporter groups into the analyte DNAs is time-consuming and expensive and may possibly be subject to technological problems.

Electrochemical methods which detect DNA-DNA interactions offer the advantage of small, robust, hand-held instruments which are suitable for what may possibly be on-site battery operation. Electrochemical determination of the DNA hybridization has thus far in the main made use of the increase in the conductivity of the double-stranded DNA after the hybridization.

Analytical methods which are based on using the conductivity of the DNA following hybridization are not well advanced technically. In these methods, powerful electric fields result in DNA damage and consequently signal loss.

In addition, the conductivity of the DNA becomes greatly reduced as the length of the double helix increases. None of the previously employed methods enables the DNA hybridization to be determined quantitatively.

What are termed redox (re)cycling tests offer a robust approach for solving the problem of making DNA hybridization accessible to an electrochemical measurement. In this approach, the hybridization event between bound catcher DNA and biotin-labeled analyte DNA is, for example, labeled by way of a biotin-streptavidin interaction using an enzyme. The activity of the biocatalyst, e.g. alkaline phosphatase, then forms a redox-active product, e.g. p-aminophenol, which can be transformed amperometrically at suitable electrodes, e.g. gold electrodes. As a result of the choice of the special electrode geometry, in particular interdigital electrodes, and of the small electrode distances of <1 μm, for example, a redox cycling process can start after suitable potentials have been applied, with the current of this process being a measure of the DNA hybridization event.

A redox cycling system is described, by way of example, in WO 01/75149 A2. In this connection, use is made, in particular, of a three-electrode system having, for example, interdigital measuring electrodes.

### SUMMARY OF THE INVENTION

An object of an embodiment of the invention is to specify an improved method for a DNA label-free biochemical analysis of the DNA-DNA interaction and to create the associated arrangements.

According to an embodiment of the invention, an object is achieved by a sequence of procedural steps. In particular, an implementation of an embodiment of the invention is termed an enzyme switch. An associated arrangement is further specified in another embodiment.

In the method according to an embodiment of the invention, it is advantageously possible to provide immobilized DNA, as catcher, with a biocatalytically active label and a substance, as inhibitor, which is able, by interaction with the label, to inhibit its catalytic activity reversibly. Alternatively, it is possible to immobilize a DNA, as catcher, in the vicinity of the immobilized biocatalytic label, with this DNA being provided with a substance, as inhibitor, which is able, by interaction with the biocatalytically active label, to inhibit its catalytic activity reversibly.

Alternatively, an immobilized biocatalytically active label can be provided with a DNA, as catcher, with this DNA as catcher, with this DNA, for its part, carrying a substance, as inhibitor, which is able, by interaction with the label, to inhibit its activity reversibly. In other alternatives, it is possible to use a complex composed of a double-stranded DNA-binding molecule and a substance, as inhibitor, which is able, by interaction with the immobilized biocatalytically active label, to inhibit its activity reversibly. When analyte DNA and immobilized catcher DNA hybridize, this complex binds to the resulting double strand and is consequently no longer available for inhibiting the biocatalytically active label.

In all the alternatives cited, the structure of the catcher DNA, i.e. its partial single-/double-strandedness, enables, in a first inactive state of the system, the inhibitor and the biocatalyst to interact. When the DNA to be analyzed binds to the catcher DNA because of the complementarity, the formation of this double strand abolishes the interaction between the biocatalyst and the inhibitor or results in the inhibitor being bound to the double strand which has formed. In this way, the system is switched from the first, inactive state into a second, active state.

An embodiment of the invention reduces or even eliminates disadvantages of the prior art. An embodiment of the invention provides, in particular, for the use of a switchable biocatalyst, namely the enzyme, with the activity of the biocatalyst being controlled and, in particular, switched by way of the hybridization of the sample DNA to the catcher DNA.

An arrangement for implementing the method according to an embodiment of the invention comprises a support, on which an enzyme is immobilized at a site, a catcher DNA which is immobilized at the site, an inhibitor which is covalently linked to the catcher DNA, and a substrate, with, in a first state, the catcher DNA being folded, by way of intramolecular hydrogen bonds, such that the inhibitor inhibits the activity of the enzyme and the substrate is not transformed, and with, in a second state, the catcher DNA hybridizing with a DNA to be detected and thereby being folded such that the inhibitor is separated from the enzyme and the substrate is transformed.

In an embodiment of the invention, the nucleic acids can be analyzed optically or electrochemically by way of a hybridization switch. In particular, the electrochemical measurement can take place amperometrically, potentiometrically or conductometrically. This thereby may result in the following substantial advantages as compared with the prior art:
a label-free read-out method for analyzing DNA is created. Thus, there is no need, for detecting the hybridization between catcher DNA and analyte DNA, to introduce any reporter group into the analyte DNA directly or indirectly by way of a further hybridization step with a signal oligonucleotide as signal DNA. This has the advantage that, when the concentration of analyte DNA is adequate, it is possible to dispense with a time-consuming and expensive PCR/SDA for introducing a label as reporter. It is also possible to do without a further hybridization, which is otherwise necessary in some cases, with a signal DNA for detecting the catcher/analyte DNA hybridization as a sandwich assay, thereby markedly simplifying the complexity of the biochemical detection system and thereby reducing sources of error.
The correlation between the quantity of enzyme product formed and the quantity of the double-stranded catcher/analyte DNA makes it possible to evaluate the analyte DNA concentration in the sample quantitatively.

### BRIEF DESCRIPTION OF THE DRAWINGS

Further advantages and details of the invention ensue from the following description of illustrated exemplary embodiments, making use of the drawing in combination with the patent claims. In each case as a diagram,

FIG. 1 shows an analytical system in accordance with the prior art,

FIGS. 2/3, 4/5, 6/7 and 8/9 in each case show systems which involve controlling the enzyme activity by means of DNA hybridization and in which a double helix is formed,

FIG. 10 shows a plan view of a transducer array together with an enlarged detail for clarifying the construction and production of the complete system,

FIG. 11 shows a scheme illustrating the course of a measurement, and

FIG. 12 shows an electrochemical system for analyzing switch functions in accordance with FIG. 2/3, 4/5, 6/7 or 8/9.

In the figures, the same elements have the same reference numbers. The figures are described below, in some cases jointly.

### DETAILED DESCRIPTION OF THE EXEMPLARY EMBODIMENTS

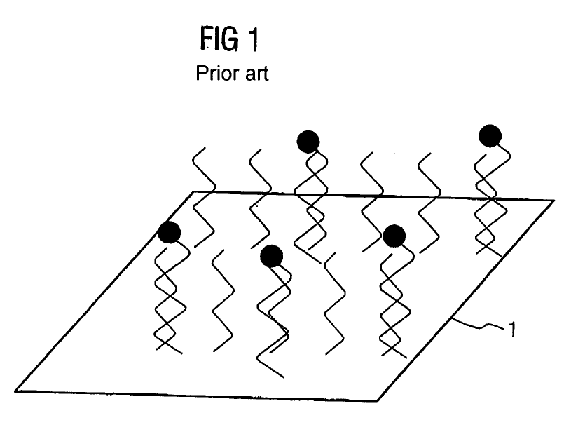

Reference has already been made to FIG. 1 in the introduction while discussing the prior art. In the case of a DNA chip which operates in accordance with the optical principle, the circles represent fluorescent reporter groups which are coupled to the analyte DNA/signal DNA. The information of interest is obtained by optical interrogation.

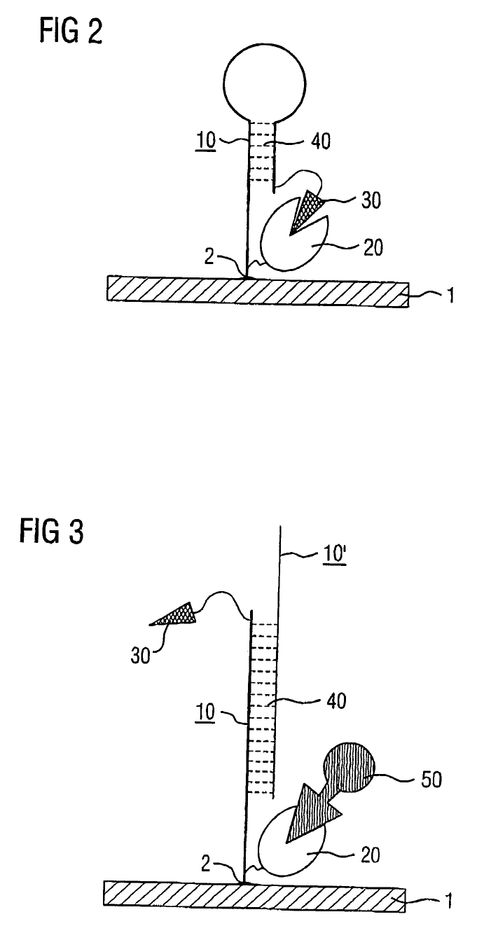

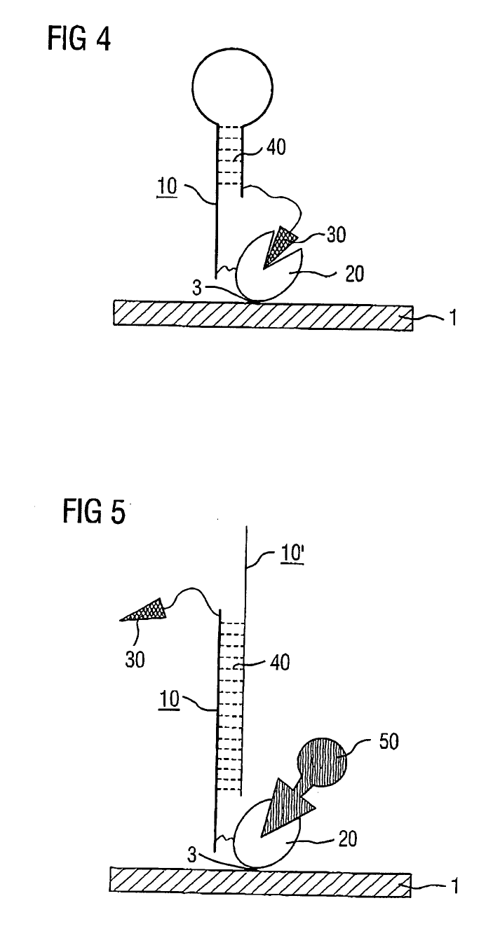

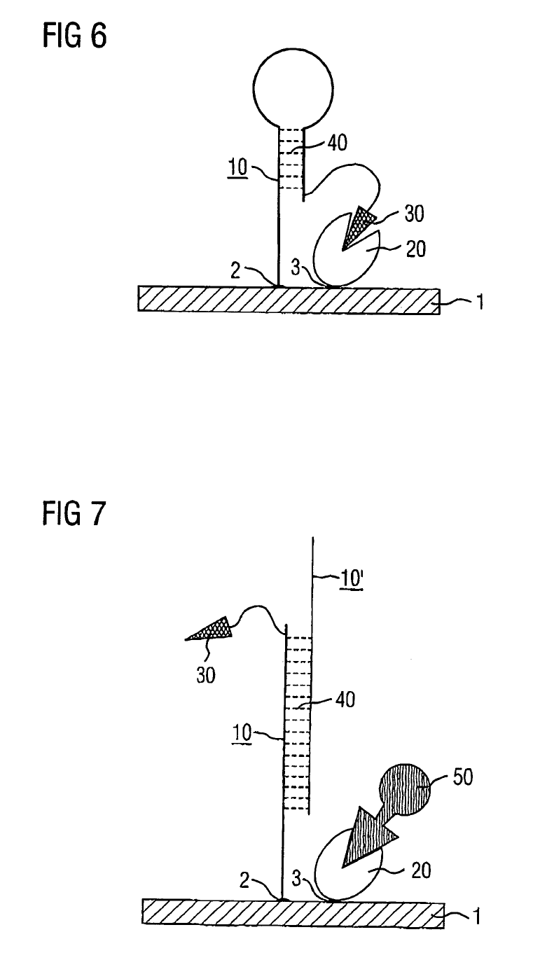

The subsequent description of the figures relates initially to FIGS. 2 to 9. The same phenomenological principles apply to all these figures.

A support 1 is in each case present as substrate in FIGS. 2 to 9. If an electrochemical read-out method, in particular redox cycling, is used, the support 1 is a chip having integrated circuits which are not shown here in detail. These circuits can be analog or digital in design.

FIGS. 2/3, 4/5, 6/7 and 8/9 in each case show control of the biocatalytic activity by means of DNA hybridization, with this consequently effecting a switch. DNA 10 or 10′, with 10 being what is termed a catcher DNA and 10′ being the DNA to be analyzed, a biocatalytically active label 20 and an inhibitor 30, whose interaction is explained below with the aid of alternative examples, are in each case present.

The biocatalytically active label 20 is, in particular, an enzyme. However, it can also be a ribozyme.

In FIGS. 2, 4 and 6, the enzyme 20 is inactive. The structure of the catcher 10, i.e. the partial intramolecular DNA double strand brought about by hydrogen bonds 40, enables the inhibitor 30 to interact with/reversibly bind to the enzyme 20, as the biocatalytically active label, and inhibit its activity. The switch is in the inactive state.

The hybridization of the catcher DNA 10 with the analyte DNA 10′ forms a DNA double strand which is composed of catcher DNA 10 and analyte DNA 10′. This takes place because the formation of this double strand is energetically more favorable, due to the higher number of base pairings, i.e. the hydrogen bonds 40, which are formed, than the formation of the partial intramolecular catcher DNA double strand, which only contains a few hydrogen bonds. The formation of this double strand brings about a conformational change in the catcher which is so powerful that the interaction of enzyme 20 and inhibitor 30 is weakened such that the inhibitor 30 comes away from the enzyme 20, with the active center of the enzyme 20 then being free and the enzyme 20 being active.

The enzyme substrate 50 which is present in the vicinity can now fill the active center of the enzyme 20. The enzyme 20 is transformed and an optically or, in particular, electrochemically, i.e. amperometrically, potentiometrically or conductometrically, detectable product will arise. The enzyme 20 is “switched-on”.

The enzyme is consequently active in FIGS. 3, 5 and 7. The switch is in the active state.

The alternatives depicted in FIGS. 2/3, 4/5, 6/7 and 8/9 relate to different variants of the binding/immobilization of biocatalytically active label and/or catcher DNA 10 and of the inhibitor 30.

In FIG. 2 and FIG. 3, both the biocatalytically active label 20 and the inhibitor 30 are bound to the catcher DNA 10, which is fixed to a site 2 on the support or chip 1.

As shown in FIG. 4 and FIG. 5, the biocatalytically active label 20 is, in an alternative to FIG. 2/3, immobilized at a site 3 on the chip 1 and both the catcher DNA 10 and the inhibitor 30 are coupled to it.

As shown in FIG. 6 and FIG. 7, the catcher DNA 10 is, in another alternative, bound to a first site 2 on the support or chip 1 while the biocatalytically active label 20 is bound to a second site 3 on the support or chip 1.

As shown in FIG. 8 and FIG. 9, the catcher DNA 10 is, in another alternative, fixed, in the inactive state, at the site 2 while the biocatalytic label is fixed at the site 3. In FIG. 8, the catcher DNA 10 is free and single-stranded because the sequence of the catcher is such that no intramolecular hydrogen bonds 40 can be formed. In contrast with the previously described alternatives, what is termed an intercalator 60, i.e. a double-stranded DNA-binding molecule, is bonded to the inhibitor 30.

In FIG. 9, an analyte DNA 10′ binds to the catcher DNA 10 with the formation of the hydrogen bonds 40. As a result of the formation of the DNA double strand, the compound or the complex composed of intercalator 60 and inhibitor 30 now binds to the double strand. The enzyme 20 is consequently freely available to the substrate 50 and is active.

In the example shown in FIG. 8/9, the enzyme 20 can also be immobilized on the support 1 and the catcher DNA 10 can be bound to it. The catcher DNA 10 can just as well be immobilized on the support 1 and the enzyme 20 can be bound to it. In this regard, the examples shown in the alternative FIGS. 6/7 and 2/3 take precedence.

In general, immobilization/integration into a polymeric gel matrix can in each case also be used as the binding of the catcher DNA 10 and/or of the bio-catalytic label 20 to the chip 1 as support. The gel matrix can be a hydrogel, which is described elsewhere.

The covalent immobilization of the biocatalytically active label, i.e. at the enzyme, is in all cases effected specifically by way of a suitable amino acid side chain. Advantageously, the enzyme possesses the following properties:
Either the product or the substrate of the enzymic reaction must be optically or amperometrically detectable. The phosphatases, esterases and proteases which catalyze the formation of phenolates and compounds of the quinone type are particularly suitable.
The enzyme should be composed of a polypeptide chain in order to ensure the immobilization of the polypeptide chains without any loss of activity.
The enzyme should be sufficiently thermostable to enable DNA-DNA hybridization to take place over wide temperature ranges. Enzymes from thermophilic organisms usually satisfy this condition. Thermo-stable enzymes can have a low specific activity at room temperatures. This problem can be solved by means of directed mutagenesis.
In order to enable the enzyme activity to be selectively immobilized and controlled over wide temperature ranges, an expression system for expressing the enzyme from a recombinant plasmid should be present.

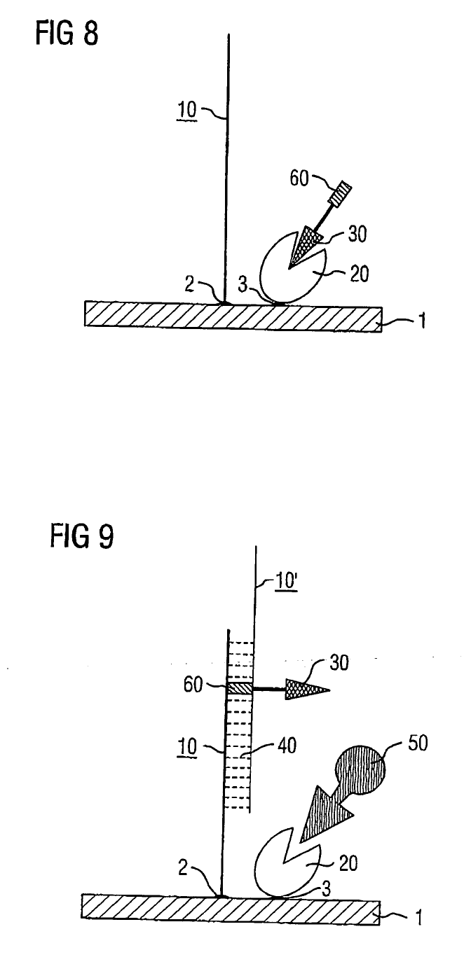

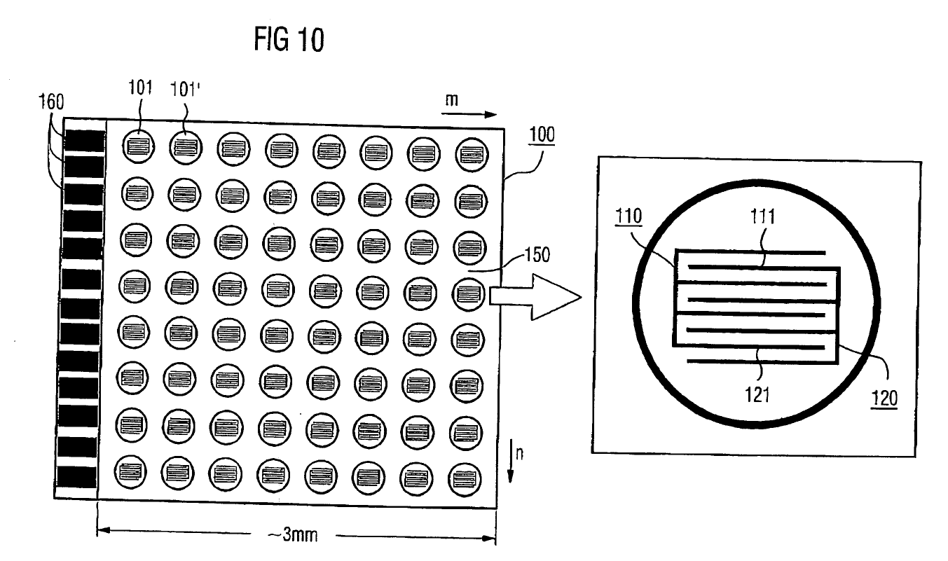

The production of a transducer system which is designed as a m×n array having m columns and n lines is described with the aid of FIG. 10: circular analytical positions 101, 101′, etc., which are separated by barriers 150, are present on a transducer surface 100 which is suitable for the redox (re)cycling method. Structures having interdigital electrodes 110 and, respectively, 120 are located on positions 101, 101′, etc., which typically have a diameter of approx. 150 μm and a distance from each other (what is termed pitch) of approx. 200 μm. The interdigital electrodes 110 and, respectively, 120 have, in a known manner, a comb-like design with electrode fingers 111 and, respectively, 121 which have a line and spacing width of not more than 1 μm and which are advantageously composed of gold. Read-out contacts 160 are arranged laterally at the transducer surface 100.

A hydrogel which is not depicted in detail and in which the catcher DNA is anchored covalently by way of a 3′ amino modification is applied to the analytical positions 101, 101′, etc. At its 5′ end, the catcher DNA carries an SH group to which the inhibitor of the reporter enzyme, e.g. carboxyl esterase, is bound covalently. An alkyl trifluoromethyl ketone, preferably a trifluoromethyl methyl ketone, is used as the reversible inhibitor of the esterase.

Catcher DNA and inhibitor are coupled in a suitable manner in accordance with the following reaction:
Oligonucleotide-5′-linker-SH+Br—CH₂—COCF₃→oligonucleotide-5′-linker-S—CH₂—COCF₃

In addition to the complex composed of catcher DNA and inhibitor, the reporter enzyme, preferably a thermostable enzyme which consists of a polypeptide chain, is anchored at each analytical position. Advantageously, the carboxyl esterase from the thermoacidophilic eubacterium Bacillus acidocaldarius (Manco, G., Adinolfi, E., Pisani, F. M., Ottolina, G. Carrera, G. and Rossi, M. 1998, Biochem. J. 332, 203-212) is chosen for this purpose. The fact that the X-ray structure of the enzyme is known (De Simone, G., Galdiero, S., Manco G., Lang, D., Rossi, M., and Pedone, C. 2000, J. Mol. Biol. 303, 761-771) is utilized for covalently binding-on the enzyme. This knowledge makes it possible to use directed mutagenesis to replace a suitable amino acid on the surface of the enzyme with cysteine or an amino acid, e.g. lysine, which has an aminofunctional radical. The enzyme is then bound directly to the gold surface of the interdigital electrodes by way of the SH group of the cysteine or else to the particular hydrogel matrix by way of the NH₂ group of the aminofunctional radical.

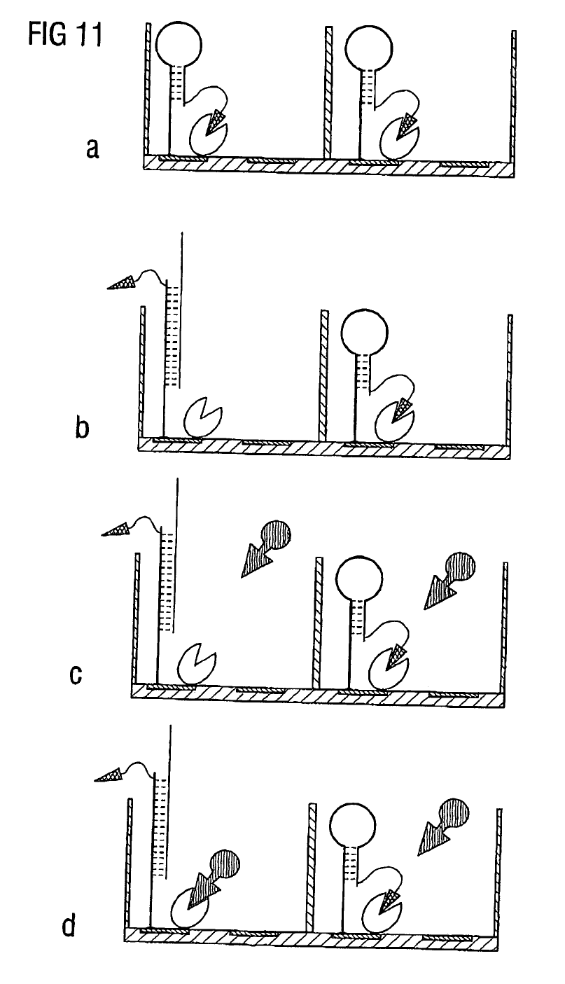

The following applies for operating the switch in accordance with the sequence scheme which is shown in FIG. 11 and which has the constituent steps a), b), c) and d): the figure shows two adjacent analytical positions which are provided with different catcher DNAs. In the ground state of the system, the catcher DNA at each respective analytical position is present, after filling with a suitable buffer solution, in a conformation where the inhibitor is able to bind to the active center of the enzyme. The enzyme is inactive; the system is correspondingly in an inactive state as shown in FIG. 11a).

After DNA to be analyzed has been added and stringent washing has taken place, a conformational change in the catcher which is so powerful that the interaction of the enzyme and inhibitor is weakened such that the inhibitor comes away from the enzyme is only brought about at the analytical position(s), specifically the left-hand of the two analytical positions in FIG. 11, where a stable nucleic acid double strand is formed as a result of the complementarity between the catcher DNA and the analyte DNA species. The active center of the enzyme is then free and the enzyme is active; the system is correspondingly in an active state as shown in FIG. 11b).

After a suitable enzyme substrate, advantageously the p-aminophenol octanoyl ester in accordance with step (c), has been added, the substrate can fill the active center of the enzyme at the analytical position(s), specifically the left-hand position in FIG. 11, at which, as can be seen from constituent FIG. 11b), a hybridization of analyte DNA and catcher DNA has taken place in accordance with step (d). It is only at these positions, specifically the left-hand position in FIG. 11, that the substrate is transformed and the amperometrically detectable product p-aminophenol can be produced.

The esterase activity is as shown in the following reaction:

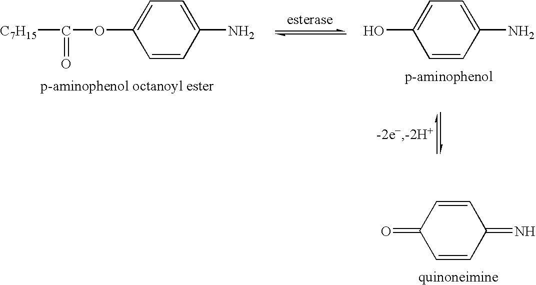

In order to amplify the signal, an oxidative or reductive potential is applied to the different “fingers” 111 and, respectively, 121 of the inter-digital electrodes 110 and, respectively, 120 of a single analytical position 101 as shown in FIG. 10. Due to the spacing and line widths, a redox cycling process then starts at the individual analytical positions at which p-aminophenol octanoyl ester has been/is being converted to p-aminophenol by means of enzymic activity. The redox cycling process is to be understood as meaning the oxidation of p-aminophenol to quinoneimine at the positively polarized electrode and the reduction of quinoneimine to p-aminophenol at the negatively polarized electrode. The total current of these redox reactions is a function of the quantity of hybridized analyte DNA.

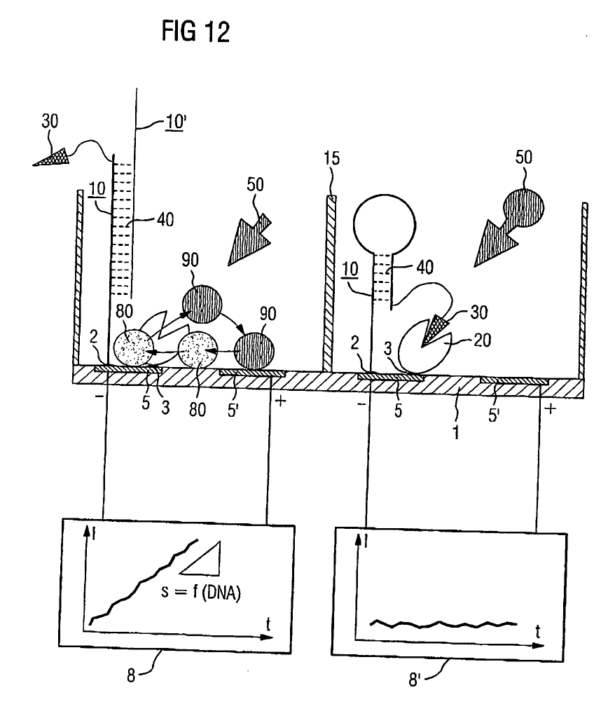

FIG. 12 clarifies the detection principle using the redox cycling process, as explained above, and the principle of electrochemical evaluation. In detail, a redox cycling process is depicted at the surface of a single analytical position 101 on the chip 1, which position is separated off by walls 15, with, in addition to the symbols which have already been explained, reference number 80 denoting the quinoneimine and reference number 90 denoting the p-amino-phenol in accordance with the above structural formula. Microelectrodes 5, 5′ are arranged on the chip 1 with a gm-spacing. The microelectrodes 5, 5′ form part of the interdigital electrodes 110 and, respectively, 120 having the finger electrodes 111 and, respectively, 121 in FIG. 10 and are supplied with different potentials. Redox currents up into the sub-nano ampere range can be measured at the microelectrodes 5, 5′ by way of measurement electronics using current meters 8 and, respectively, 8′.

A time-dependent measurement signal I=g(t), whose slope S=f(DNA) depends on the DNA to be analyzed, is obtained. This thereby creates a procedure which can be used to evaluate the DNA electrochemically. The essential advantage of this procedure is that it makes it possible to use DNA samples which have not previously been modified with a label.

The adjacent measurement positions in FIG. 12 correspond to the single analytical positions 101, 101′, etc. as shown in FIG. 10. As described in detail in connection with the latter figure, they typically have a 200 μm grid size, which means that a large number of parallel measurements can be carried out on one chip 1.

The above-described examples can be used to produce microchips for a hand-held instrument which is simple to operate and which can be used for the defined applications. The replaceable chips have a defined lifetime and can be programmed with different catchers. Since this DNA chip type is a disposable product, a requirement for very high numbers of different DNA chips can be expected. No comparable, simple-to-use instruments of this type exist on the market.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for detecting DNA with use being made of a system employing immobilized DNAs as an analytical tool, comprising:
fixing the enzyme in a stationary manner in the system which is used as an analytical tool and which contains a catcher DNA, an inhibitor and an enzyme, as biocatalytic label;
using the catcher DNA to permit, in a first inactive state of the system, the inhibitor and enzyme to interact;
forming, when the DNA to be analyzed is bound to the catcher DNA, a double strand due to the complementarity and at least one of abolishing and preventing the interaction between enzyme and inhibitor, wherein
in this way, the system is switched from the first inactive state to a second, active state; and
measuring the signals of at least one of the active and inactive state via an electrochemically detectable substance whose concentration changes due to the enzyme activity.

2. The method as claimed in claim 1, wherein the structure of the catcher DNA enables the inhibitor and biocatalyst to interact.

3. The method as claimed in claim 2, wherein immobilized DNA is used as catcher DNA and wherein the immobilized DNA is provided with the biocatalytically active label and, as inhibitor, a substance which is able, by interaction with the label, to inhibit its catalytic activity reversibly.

4. The method as claimed in claim 2, wherein an immobilized biocatalytic label is used and wherein a DNA, which is provided with a substance, as inhibitor, which is able, by interaction with the label, to inhibit its catalytic activity reversibly, is immobilized, as catcher, in the vicinity of the immobilized biocatalytic label.

5. The method as claimed in claim 2, wherein an immobilized biocatalytic label is used and wherein the immobilized biocatalytically active label is provided with a DNA, as catcher, which DNA, for its part, carries a substance, as inhibitor, which is able, by interaction with the label, to inhibit its activity reversibly.

6. The method as claimed in claim 1, wherein use is made of a complex composed of a double-stranded DNA-binding molecule and an inhibitor substance which is able, by interaction with the immobilized biocatalytically active label, to inhibit its activity reversibly, with the complex being bound, when the immobilized catcher DNA and the analyte DNA hybridize, to the resulting double strand and consequently no longer being available for inhibiting the biocatalytically active label.

7. The method as claimed in claim 1, wherein the inhibitor is a substance which binds reversibly to the enzyme and inhibits the enzymic activity.

8. The method as claimed in claim 1, wherein the DNA to be analyzed forms, by hybridization with the immobilized DNA, a double strand, i.e. a double helix, on account of the complementarity of the single-stranded DNAs.

9. The method as claimed in claim 1, wherein the first, inactive state and the second, active state of the system, and its change from_the first state to the second state, together constitute a switching function.

10. The method as claimed in 1, wherein the switching function of the system is effected by the DNA to be analyzed hybridizing to the immobilized DNA, as catcher.

11. The method as claimed in claim 9, wherein the state of the switching function of the system is interrogated by determining the activity of the biocatalyst.

12. The method as claimed in claim 1, further-comprising:
using an enzyme;
forming an activatable switch from the enzyme; and
reading out the signal of the enzyme switch least one of optically and electrochemically.

13. The method as claimed in claim 12, wherein the enzyme switch is controlled by the catcher DNA and the DNA to be analyzed hybridizing under stringent conditions.

14. The method as claimed in claim 12, wherein a product which is at least one optically and electrochemically detectable is synthesized, and wherein the enzyme catalyzes the conversion of an undetectable substrate into a product at least one of optically and electrochemically detectable.

15. The method as claimed in claim 12, wherein the electrochemical read-out is effected at least one of amperometrically, potentiometrically and conductometrically.

16. The method as claimed in claim 12, wherein the measured values of the enzyme switch are output digitally and can be read off directly.

17. The method as claimed in claim 12, wherein the analyte DNA concentration is effected by correlation between the quantity of enzyme product released and the quantity of DNA to be analyzed which is hybridized.

18. The method as claimed in claim 17, wherein the enzyme switch is deactivated by interaction of the inhibitor with the enzyme.

19. The method as claimed in claim 18, wherein the enzyme is inactivated if the inhibitor is bound to the enzyme and in that the inhibitor is unavailable to the enzyme, because of the double strand, and the enzyme is active, when a double helix exists between the catcher DNA and the DNA to be analyzed.

20. An arrangement, comprising:
a support on which an enzyme is immobilized at a site;
a catcher DNA which is immobilized at the site;
an inhibitor which is covalently linked to the catcher DNA and a substrate, with, in a first state, the catcher DNA being folded by way of intramolecular hydrogen bonds such that the inhibitor inhibits the activity of the enzyme and the substrate is not transformed, and with, in a second state, the catcher DNA hybridizing with a DNA to be detected and thereby being folded such that the inhibitor is separated from the enzyme and the substrate is transformed, wherein the support includes integrated microelectrodes, with the enzyme being at least one of immobilized on the support, and being at least one of enclosed and immobilized in a polymer network in the vicinity of the microelectrodes, and wherein at least one of the product and the substrate of the enzymic reaction is electrochemically detectable at the microelectrodes.

21. The arrangement as claimed in claim 20, wherein the polymer network does not interfere with the activity of the enzyme and is permeable for the analyte DNA to be analyzed.

22. The arrangement as claimed in claim 20, wherein the enzyme is at least one of a phosphatase, esterase and protease.

23. The arrangement as claimed in claim 22, wherein the enzyme is composed of a polypeptide chain and wherein the polypeptide chain is immobilized without the enzyme losing any activity.

24. The arrangement as claimed in claim 23, wherein the enzyme is thermostable.

25. The arrangement as claimed in claim 24, wherein the enzyme can be produced by an expression system which comprises at least one recombinant plasmid.

26. (canceled)

27. (canceled)

28. The method as claimed in claim 1, wherein the partial double/single strandedness of the catcher DNA enables the inhibitor and biocatalyst to interact.

29. The method as claimed in claim 13, wherein a product which is at least one optically and electrochemically detectable is synthesized, and wherein the enzyme catalyzes the conversion of an undetectable substrate into a product at least one of optically and electrochemically detectable.

30. The method as claimed in claim 13, wherein the electrochemical read-out is effected at least one of amperometrically, potentiometrically and conductometrically.

31. The method as claimed in claim 14, wherein the electrochemical read-out is effected at least one of amperometrically, potentiometrically and conductometrically.

32. The apparatus of claim 20, wherein at least one of the product and the substrate of the enzymic reaction is at least one of amperometrically, potentiometrically and conductometrically detectable at the microelectrodes.
